(19) United States
(12) Patent Application Publication
(10) Pub. No.: US 2026/0144662 A1
(43) Pub. Date: May 28, 2026

(54) JOINT FOR ORTHOTIC DEVICES AND ORTHOTIC DEVICE

(71) Applicant: Izumi Prosthetics and Orthotics Manufacturing Co., Ltd., Izumi-shi (JP)

(72) Inventor: Yuji YUMIKINO, Izumi-shi (JP)

(21) Appl. No.: 19/362,399

(22) Filed: Oct. 19, 2025

(30) Foreign Application Priority Data
Nov. 25, 2024 (JP) 2024-204730

(51) Int. Cl. A61F 5/01

(52) U.S. Cl. CPC A61F 5/013; A61F 2005/0155; A61F 2005/0165; A61F 2005/0179

(57) ABSTRACT

The joint portion 4 applied to an orthotic device 1 comprises a first connecting portion 41, a second connecting portion 42, and a shaft support portion 5. The shaft support portion 5 features a ratchet gear 51 with multiple ratchet teeth 512 formed on its outer periphery. These teeth consist of first ratchet teeth 512a, inclined in one direction on one half of the periphery, and second ratchet teeth 512b, inclined in the opposite direction on the other half, divided by a diameter line perpendicular to the rotation axis. A first stopper 52a and a second stopper 52b engage with these teeth 512. By operating either one of the stoppers to release its engagement, the joint portion 4 can be rotated in a predetermined direction.

### TECHNICAL FIELD

The present invention relates to a joint for orthotic devices, and relates to an orthotic device. Specifically, the present invention relates to a rehabilitation orthotic device that is used to improve a symptom of restricted articular movement caused by encephalopathy or by brain damage, and relates to a joint for orthotic devices and an orthotic device that have a simple structure and that are low in cost and are practicable for a patient.

### DESCRIPTION OF THE RELATED ART

When contracture, paralysis, muscular atrophy, and the like (hereinafter collectively referred to as “contracture and the like” if necessary) of fingers or of a wrist occur due to encephalopathy, brain damage, and the like including cerebral stroke, cerebral hemorrhage, and cerebral infarction, a patient cannot freely move fingers or articulations, and palmar flexion occurs on the wrist, and as a result, a state in which the fingers have been tightly squeezed is reached.

Then, as a result of continuance of this state, the patient is wound by his/her own nails in the palm, or a fester and a pressure sore are caused by pressure between the skins, or dirty substances gather, which are undesirable from the viewpoint of sanitation. Therefore, in order to enable the patient to smoothly perform daily life as soon as possible, rehabilitation is required which includes range-of-articulation-motion exercises for improving articulation motion at an early stage after the onset of encephalopathy.

In the range-of-articulation-motion exercises, an articulated joint is employed as an orthotic device, and the commercially available articulated joint is a fixed type joint that has no range of motion or, is a movable type joint that is moved with a predetermined angular interval and that is fixed by a bolt, a stopper, or the like at a predetermined angular position for range-of-motion exercises. Additionally, a shift is brought from an acute stage to a recovery stage in accordance with the progression of rehabilitation, and it is necessary to substitute the fixed type joint for the movable type joint that is used for range-of-motion exercises along with the improvement of articulation rigid motion by range-of-motion exercises.

For example, Patent Document 1 discloses a hand orthotic device that is composed of a glove-shaped hand interface that is worn on a palm and a control module that has an actuator including a motor or the like used to supply torque to the hand interface.

According to Patent Document 1, even a patient who cannot move the articulations by his/her will can easily perform articulation motion by transmitting a linear force generated by the control module as rotational torque to the finger interface through a wire. Additionally, rehabilitation according to each of the acute stage and the recovery stage becomes possible by controlling the transmission of torque from the control module to the finger interface.

### PRIOR ART DOCUMENT

### Patent Document

[Patent Document 1] Japanese translation of PCT international application 2022-505441

### SUMMARY OF THE INVENTION

### Problem to Be Solved by the Invention

However, the hand orthotic device disclosed by Patent Document 1 described above is heavy in weight, and therefore, for example, it was very difficult for a person having an upper-limb disability to wear the hand orthotic device on the palm or arm by him/herself, and the person was unable to easily use the hand orthotic device. Furthermore, a problem has resided in the fact that the hand orthotic device has a complicated structure, and is very high in cost.

The present invention has been made in view of the aforementioned circumstances, and it is an object of the present invention to provide a rehabilitation orthotic device that is used to improve a symptom of restricted articular movement caused by encephalopathy or by brain damage, and a joint for orthotic devices and an orthotic device that have a simple structure and are low in cost and are practicable for a patient.

### Means for Solving the Problem

In order to achieve the aforementioned object, a joint for orthotic devices of the present invention includes: a first connecting portion that includes a first base end portion and a first head portion, the first base end portion being connected to a first member; a second connecting portion that includes a second base end portion and a second head portion, the second base end portion being connected to a second member; and a shaft support portion that pivotally supports the first head portion of the first connecting portion and the second head portion of the second connecting portion with respect to each other so as to be rotatable in one direction and in one other direction opposite to the one direction at a position substantially corresponding to an articulation, and that has a ratchet gear including a plurality of ratchet teeth formed along an outer periphery of a substantially circular-shaped main body portion, a first stopper that engages with the ratchet teeth and that restricts rotation in one direction, and a second stopper that engages with the ratchet teeth and that restricts rotation in one other direction.

Herein, the first connecting portion can be connected to the first member that is a constituent of an orthotic device by providing the first connecting portion that includes the first base end portion and the first head portion, with the first base end portion connected to the first member.

Additionally, the second connecting portion can be connected to the second member that is a constituent of the orthotic device by providing the second connecting portion that includes the second base end portion and the second head portion, with the second base end portion connected to the second member.

Additionally, the shaft support portion enables the second member with respect to the first member or the first member with respect to the second member to rotate in one direction and in one other direction by providing the shaft support portion that pivotally supports the first head portion of the first connecting portion and the second head portion of the second connecting portion with respect to each other so as to be rotatable in one direction and in one other direction opposite to the one direction at a position substantially corresponding to the articulation.

Additionally, the shaft support portion includes a first stopper that engages with the ratchet teeth and restricts rotation in one direction and a second stopper that engages with the ratchet teeth and restricts rotation in one other direction, and, as a result, for example, rotation in the one other direction is permitted by engaging the first stopper with the ratchet teeth and by releasing an engagement state between the second stopper and the ratchet teeth, and rotation in the one direction is permitted by engaging the second stopper with the ratchet teeth and by releasing an engagement state between the first stopper and the ratchet teeth. Furthermore, the rotation of the ratchet gear itself can be restricted by simultaneously engaging the first stopper and the second stopper with the ratchet teeth. Therefore, the state of the orthotic device can be freely changed according to a rehabilitation situation of a patient wearing the orthotic device.

Additionally, the ratchet gear is sandwiched between the first head portion and the second head portion, the ratchet teeth includes a first ratchet tooth inclined in one rotation direction in a half peripheral portion on one side of the main body portion on a boundary of a diameter line substantially orthogonal to a rotation axis of the ratchet gear, and a second ratchet tooth inclined in one other rotation direction in a half peripheral portion on the other side of the main body portion, and when the first stopper is engaged with the first ratchet tooth and when the second stopper is engaged with the second ratchet tooth, an engagement state between the first and second stoppers and the first and second ratchet teeth is switched, and as a result, rotation in the one direction or in the one other direction can be easily performed.

Additionally, in a case in which the first head portion or the second head portion is provided with the first stopper holding portion that holds the first stopper and the second stopper holding portion that holds the second stopper, the first stopper and the second stopper can be held by the respective holding portions, and therefore there is no possibility that the stopper will be lost.

Additionally, when the first stopper holding portion houses the first spring that urges the first stopper in a direction in which the first stopper is engaged with the ratchet teeth and houses the first wire that is connected to the first stopper and that applies a tensile force in a direction against the urging force of the first spring by an external operation, the first stopper normally engages with the first ratchet tooth so as to regulate rotation in the one direction, and only when rotation in the one direction is intended, the first wire is operated so as to release the engagement state between the first stopper and the first ratchet tooth, and rotation in the one direction can be permitted.

Additionally, when the second stopper holding portion houses the second spring that urges the second stopper in a direction in which the second stopper is engaged with the second ratchet tooth and houses the second wire that is connected to the second stopper and that applies a tensile force in a direction against the urging force of the second spring by an external operation, the second stopper normally engages with the second ratchet tooth so as to regulate rotation in the one other direction, and only when rotation in the one other direction is intended, the second wire is operated so as to release the engagement state between the second stopper and the second ratchet tooth, and rotation in the one other direction can be permitted.

Additionally, in a case in which the first connecting portion includes the rotation restricting portion that restricts the rotation range in one direction and one other direction of the second connecting portion, the rotation range of the second member can be restricted in accordance with the movement of the articulation of a patient wearing the orthotic device by restricting the rotation range of the second connecting portion.

Additionally, in a case in which a concave portion in which ratchet teeth are loosely fitted is formed on either one of the first and second head portions, and a protrusion portion that fixes the ratchet teeth is provided on the other one of the first and second head portions, and the first head portion and the second head portion are fitted together so as to form the same plane, it is possible to install the joint for orthotic devices even in a narrow space by eliminating a projection of an overlapping surface between the first head portion and the second head portion. Additionally, the shaft support portion cannot be seen from the outside, and therefore the appearance is excellent, and it is possible to prevent breakage of the shaft support portion when an external force acts.

Additionally, when the angle formed by an imaginary straight line connecting the vertex of the first ratchet tooth and the rotation axis of the ratchet gear and the inclined surface of the front tooth of the first ratchet tooth is approximately 40° to 45°, and when the angle formed by an imaginary straight line connecting the vertex of the second ratchet tooth and the rotation axis of the ratchet gear and the inclined surface of the front tooth of the second ratchet tooth is approximately 40° to 45°, the shaft support portion can rotate most smoothly, and wear caused by use can be reduced.

In order to achieve the aforementioned object, an orthotic device according to the present invention includes: a forearm wearing portion that is worn on a forearm; a hand wearing portion that is worn on a hand; and a joint for orthotic devices that pivotally supports the hand wearing portion rotatably with respect to the forearm wearing portion, the joint for orthotic devices including: a first connecting portion including a first base end portion and a first head portion, the first base end portion being connected to a first forearm wearing portion; a second connecting portion including a second base end portion and a second head portion, the second base end portion being connected to the hand wearing portion; and a shaft support portion, the shaft support portion including: a ratchet gear pivotally supporting the first head portion of the first connecting portion and the second head portion of the second connecting portion with respect to each other so as to be rotatable in a palmar flexion direction and in a dorsiflexion direction at a position substantially corresponding to a wrist joint and including a plurality of ratchet teeth formed along an outer periphery of a substantially circular-shaped main body portion; a first stopper that engages with the ratchet teeth and that restricts rotation in the dorsiflexion direction; and a second stopper that engages with the ratchet teeth and that restricts rotation in the palmar flexion direction.

Herein, the joint for orthotic devices includes the first connecting portion including the first base end portion and the first head portion and in which the first base end portion is connected to the forearm wearing portion, and therefore the first connecting portion can be connected to the forearm wearing portion that is a constituent of the orthotic device.

Additionally, the joint for orthotic devices includes the second connecting portion including the second base end portion and the second head portion and in which the second base end portion is connected to the hand wearing portion, and therefore the second connecting portion can be connected to the hand wearing portion that is a constituent of the orthotic device.

Additionally, the shaft support portion enables the hand wearing portion with respect to the forearm wearing portion to rotate in the palmar flexion direction and in the dorsiflexion direction by providing the shaft support portion that pivotally supports the first head portion of the first connecting portion and the second head portion of the second connecting portion with respect to each other so as to be rotatable in the palmar flexion direction and in the dorsiflexion direction at a position substantially corresponding to the wrist joint. Therefore, it is possible to efficiently perform rehabilitation of the wrist joint caused by encephalopathy or brain damage by wearing the orthotic device mentioned above.

Additionally, the first stopper that engages with the ratchet teeth and that restricts rotation in the dorsiflexion direction and the second stopper that engages with the ratchet teeth and that restricts rotation in the palmar flexion direction are provided, and therefore, for example, the first stopper is engaged with the ratchet teeth, and the engagement state between the second stopper and the ratchet gear is released, so that the rotation in the palmar flexion direction is permitted. On the other hand, the second stopper is engaged with the ratchet teeth, and the engagement state between the first stopper and the ratchet gear is released, so that the rotation in the dorsiflexion direction is permitted. Furthermore, the rotation of the ratchet gear itself can be restricted by simultaneously engaging the first stopper and the second stopper with the ratchet teeth. Therefore, the state of the orthotic device can be freely changed according to a rehabilitation situation of a patient wearing the orthotic device.

### Advantageous Effects of the Invention

The joint for orthotic devices and the orthotic device according to the present invention have a simple structure and are low in cost and are practicable for a patient.

### BRIEF DESCRIPTION OF THE DRAWINGS

FIGS. 1A and 1B show a state in which an orthotic device according to an embodiment of the present invention has been worn on an affected limb, and FIG. 1A is a side view, and FIG. 1B is a plan view.

FIGS. 2A and 2B are enlarged views of a main part of a joint portion according to an embodiment of the present invention, and FIG. 2A is an assembly view of the main part of the joint portion, and FIG. 2B is an exploded view of the main part of the joint portion.

FIGS. 3A and 3B are views showing a state in which the orthotic device according to an embodiment of the present invention has been worn on the affected limb, and has been turned in a dorsiflexion direction.

FIGS. 4A and 4B are views showing a state in which the orthotic device according to an embodiment of the present invention has been worn on the affected limb, and has been turned in a palmar flexion direction.

### MODES FOR CARRYING OUT THE INVENTION

Hereinafter, to understand the present invention, embodiments of the present invention relating to a joint for orthotic devices (hereinafter referred to as a “joint portion”) and an orthotic device will be described with reference to the drawings. Note that, in each of the drawings, for descriptive convenience, a state in which the arm, to which an orthotic device 1 is worn such that the back of the hand is vertically upwardly directed, is horizontally extended in parallel with the ground, is defined as a reference state, and the direction pointed upwardly from the back of the hand direction is defined as an upward direction, and the direction opposite to the upward direction is defined as a downward direction, and the axial direction represented by the upward direction and the downward direction is defined as a vertical axis, and the axial direction perpendicular to the vertical axis is defined as a horizontal axis, and the back-of-the-hand side is referred to as a front surface if necessary, and the palmar side opposite to the back-of-the-hand side is referred to a rear surface if necessary. Additionally, in the following embodiment, specifications according to which the orthotic device 1 is worn on a patient's left hand will be described, and the same applies to a case in which the orthotic device 1 is worn on a patient's right hand.

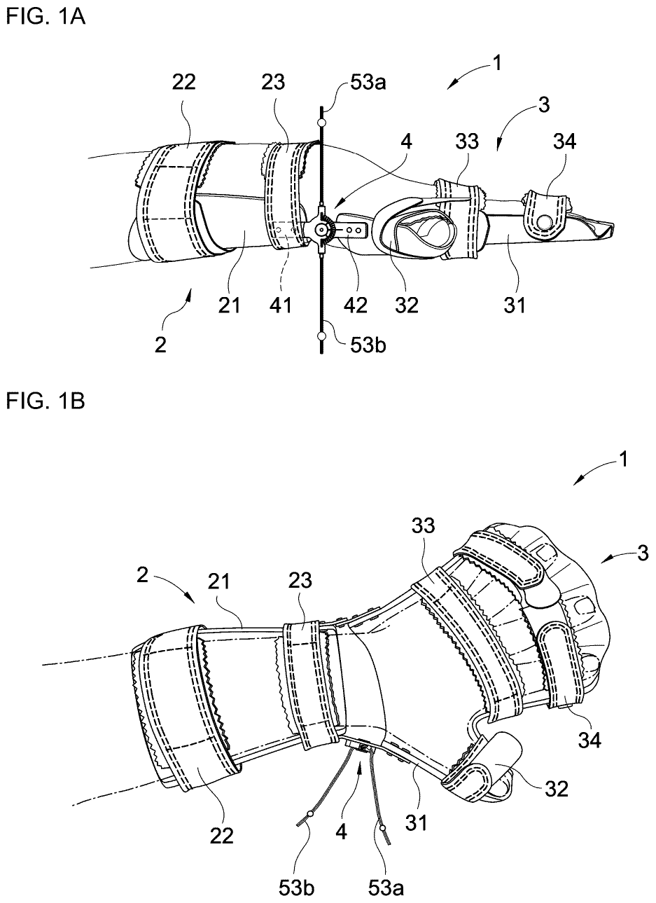

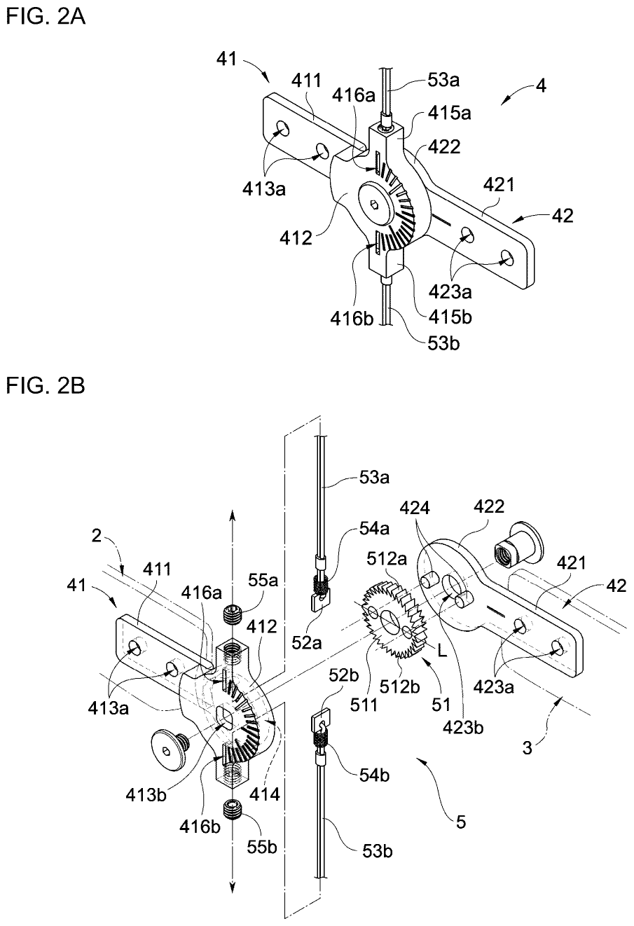

First, an overall configuration of the orthotic device 1 according to the embodiment to which the present invention is applied will be described with reference to FIGS. 1A to 2B. The orthotic device 1 is made of, for example, a synthetic resin material or the like, and is composed mainly of a forearm wearing portion 2, a hand wearing portion 3, and a joint portion 4.

### Forearm Wearing Portion

The forearm wearing portion 2 has a length from the elbow to just before the wrist joint, and has a substantially U-shaped cross section that is worn on the rear surface of the forearm and covers from the radius to the ulna, and includes a main body portion 21 and a first belt body 22 and a second belt body 23 that are used to fix the main body portion 21 to the forearm of the arm on which the orthotic device is worn in a state in which the main body portion 21 is worn on the rear surface of the forearm. Then, the main body portion 21 is fixed by a fixing means, such as a hook-and-loop fastener, by winding the first belt body 22 and the second belt body 23 in the circumferential direction of the upper arm in a state in which the main body portion 21 has been worn on the rear surface of the forearm.

Herein, the forearm wearing portion 2 is not necessarily required to be worn only on the rear surface of the forearm, and may be configured to cover the entire forearm.

Additionally, a method of fixing the forearm wearing portion 2 to the forearm is not necessarily required to be executed by the first belt body 22 and the second belt body 23 described above, and other known fixing methods may be adopted.

### Hand Wearing Portion

The hand wearing portion 3 is worn at the palmar side, and has a main body portion 31 imitating the shape of the hand, and additionally has a belt body that is used to fix the hand in a state in which the hand is placed on the main body portion 31. The belt body includes a third belt body 32 that winds around the thumb, a fourth belt body 33 that fixes the palm, and a fifth belt body 34 that fixes the four fingers other than the thumb. Then, the hand wearing portion 3 is fixed by a fixing means, such as a hook-and-loop fastener, by winding the third belt body 32, the fourth belt body 33, and the fifth belt body 34 in the circumferential direction in a state in which the hand wearing portion 3 has been worn on the palmar side.

Herein, the hand wearing portion 3 is not necessarily required to be worn on the palmar side, and may be configured to be worn on the back-of-the-hand side.

Additionally, a method of fixing the hand wearing portion 3 to the hand is not necessarily required to be executed by the third belt body 32, the fourth belt body 33, and the fifth belt body 34 described above, and other known fixing methods may be adopted.

### Joint Portion

The joint portion 4 connects the forearm wearing portion 2 and the hand wearing portion 3 together so that the hand wearing portion 3 is rotatable with respect to the forearm wearing portion 2 in conjunction with the movement of the wrist in the palmar flexion direction and the dorsiflexion direction, and the joint portion 4 includes a first connecting portion 41, a second connecting portion 42, and a shaft support portion 5, and the shaft support portion 5 is located on the radial side so as to substantially correspond to the wrist joint.

Herein, the joint portion 4 is not necessarily required to be located on the radial side, and may be located at a position substantially corresponding to the wrist joint on the ulnar side opposite to the radial side. Additionally, the joint portion 4 may be located at positions substantially corresponding to the wrist joint on the radial side and the wrist joint on the ulnar side, respectively. However, the joint portion 4 is located on the radial side, and therefore the patient can easily observe the condition of the joint portion 4 when performing the motion in the palmar flexion direction or the dorsiflexion direction in the reference state, and, in consideration of setting an arbitrary angle by operating the shaft support portion 5 as described later, the shaft support portion 5 is preferably located on the radial side.

As shown in FIGS. 2A and 2B, the first connecting portion 41 includes a first base end portion 411 and a first head portion 412 connected to the first base end portion 411. A pair of through holes 413a are formed in the first base end portion 411, and the first base end portion 411 is fixed by bolt nuts (not shown) using these through holes 413a in a state of being aligned with a forward edge portion of the forearm wearing portion 2.

In the first head portion 412, a circular concave groove 414 in which a ratchet gear 51 (described below) can be loosely fitted is formed, and a through hole 413b through which a bolt nut (not designated by a reference sign) passes is formed substantially at the center. Additionally, a first stopper holding portion 415a and a second stopper holding portion 415b both of which hold a first stopper 52a and a second stopper 52b engaged with a ratchet teeth 512 of the ratchet gear 51, respectively, are located around the first head portion 412 so as to protrude in the radial direction of the first head portion 412 and so as to face each other in the vertical direction.

The first stopper holding portion 415a has a substantially square cylindrical shape with a hollow inside, and a first slit 52a with which a first stopper 416a is engaged is formed with a predetermined length in the height direction from the base end, and a first fastener 55a that prevents a first spring 54a, a first wire 53a, and a first spring 54a constituting the shaft support portion 5 (described later) from jumping out to the outside is housed inside.

The second stopper holding portion 415b has the same shape as the first stopper holding portion 415a, and has a substantially rectangular cylindrical shape with a hollow inside, and a second slit 416b with which the second stopper 52b is engaged is formed with a predetermined length in the height direction from the base end, and a second fastener 55b that prevents a second spring 54b, a second wire 53b, and a second spring 54b constituting the shaft support portion 5 (described later) from jumping out to the outside is housed inside.

The second connecting portion 42 is composed of a second base end portion 421 and a second head portion 422 connected to the second base end portion 421. A pair of through holes 423a are formed in the second base end portion 421, and the second base end portion 421 is fixed by bolt nuts (not shown) using these through holes 423a in a state of being aligned with a rearward edge portion of the hand wearing portion 3. The second head portion 422 has a pair of ridge portions 424 that are used to insert and fix the ratchet gear 51 (described later), and a through hole 423b that is used to fix the first head portion 412 described above by a bolt nut in a state of coinciding with the first head portion 412 is formed substantially at the center.

Herein, the first stopper holding portion 415a and the second stopper holding portion 415b are not necessarily required to be located at the first head portion 412, and may be located at the second head portion 422. Additionally, the first stopper holding portion 415a and the second stopper holding portion 415b can be located at the first head portion 412 and the second head portion 422, respectively, or vice versa.

### Shaft Support Portion

Next, the shaft support portion 5 will be described with reference to FIG. 2B. The shaft support portion 5 is composed mainly of the ratchet gear 51, a stopper 52 that engages with the ratchet teeth 512 of the ratchet gear 51 to restrict the rotation of the ratchet gear 51, a wire 53 that is used to perform switching between an engagement state and a disengagement state between the stopper 52 and the ratchet teeth 512, a spring 54 that urges the stopper 52 toward the side of the ratchet gear 51 in a stopper holding portion 415, and a fastener 55 that prevents the spring 54 from jumping out of the stopper holding portion.

The ratchet gear 51 has a substantially circular-shaped main body portion 511 made of a metal material and having a thickness of 2 to 3 mm, and a plurality of ratchet teeth 512 are formed along an outer periphery of the main body portion 511. In the ratchet teeth 512, in a horizontal state, a plurality of first ratchet teeth 512a inclined at an inclination angle of approximately 40° to 45° in the dorsiflexion direction are uniformly formed in an upper half peripheral portion with a diameter line L in the horizontal direction substantially orthogonal to the rotation axis of the ratchet gear 51 as a boundary, and a plurality of second ratchet teeth 512b inclined at an inclination angle of approximately 40° to 45° in the palmar flexion direction are uniformly formed in a lower half peripheral portion.

Note that, when the inclined surface facing the outside is defined as the front tooth and the inclined surface facing the inside opposite thereto is defined as the rear tooth, the definition of the inclination angle of the ratchet tooth 512 is an angle formed by an imaginary straight line connecting the vertex of the ratchet tooth 512 and the rotation axis of the ratchet gear 51 and the inclined surface of the front tooth of the ratchet tooth 512.

Herein, as a result of examination by the inventor, in a case in which the inclination angle of the ratchet tooth 512 is less than approximately 40°, the engagement state of the stopper 52 with respect to the ratchet tooth 512 is weakened, and thus, there is a possibility that the engagement state will be unintentionally canceled when an external force acts on the orthotic device 1. On the other hand, when the inclination angle of the ratchet tooth 512 exceeds 45°, the frictional resistance with the stopper 52 when the ratchet tooth 512 rotates in one direction increases, and there is a concern that the progress of wear of the ratchet tooth 512 or the stopper 52 will be accelerated.

Therefore, from the viewpoint of maintaining the engagement state between the ratchet tooth 512 and the stopper 52 constant and enhancing the durability of the ratchet tooth 512 and the stopper 52, the inclination angle of the ratchet tooth 512 is preferably 40° to 45°, and yet the inclination angle of the ratchet tooth 512 can be arbitrarily changed depending on the thickness, size, and material of the ratchet tooth 512 or the thickness, size, and material of the stopper 52.

The first stopper 52a is made of a metallic rectangular plate-shaped body having a thickness of 2 to 3 mm, and as described above, the first stopper 52a is held in a state of being inserted into the first slit 416a formed in the first stopper holding portion 415a, and the first wire 53a is engaged with a through hole (not designated by a reference sign) formed in the first stopper 52a. Then, the first wire 53a is inserted through the first spring 54a and the first fastener 55a, and extends from the first stopper holding portion 415a to the outside. Note that, the first fastener 55a is a screw member having a hollow inside and a screw groove threaded on the outer periphery, and is fixedly screwed to a screw groove formed on the inner periphery of the first stopper holding portion 415a.

Additionally, the second stopper 52b is made of a metallic rectangular plate-shaped body having a thickness of 2 to 3 mm, and is held in a state of being inserted into the second slit 415b formed in the second stopper holding portion 416b, and the second wire 53b is engaged with a through hole (not designated by a reference sign) formed in the second stopper 52b. Then, the second wire 53b is inserted through the second spring 54b and the second fastener 55b, and extends from the second stopper holding portion 415b to the outside. Additionally, the second fastener 55b also has the same configuration as the first fastener 55a, and is a screw member having a hollow inside and a screw groove threaded on the outer periphery, and is fixedly screwed to a screw groove formed on the inner periphery of the second stopper holding portion 415b.

Herein, each of the first stopper 52a and the second stopper 52b is not necessarily required to be rectangular plate-shaped body. The shapes of the first and second stoppers 52a and 52b are not particularly limited as long as the first and second stoppers 52a and 52b can be engaged with the ratchet teeth 512.

Additionally, the first fastener 55a and the second fastener 55b are not limited to the configurations described above, and may have any configuration as long as the first spring 54a and the second spring 54b do not protrude to the outside from the first stopper holding portion 415a and the second stopper holding portion 415b.

The first spring 54a and the second spring 54b are constantly urged toward the ratchet teeth 512, respectively. Therefore, in the normal state, the first stopper 52a held by the first stopper holding portion 415a is engaged with the first ratchet teeth 512a, and the second stopper 52b held by the second stopper holding portion 415b is engaged with the second ratchet teeth 512b, so that the turning of the hand wearing portion 3 in the palmar flexion direction and the dorsiflexion direction is restricted.

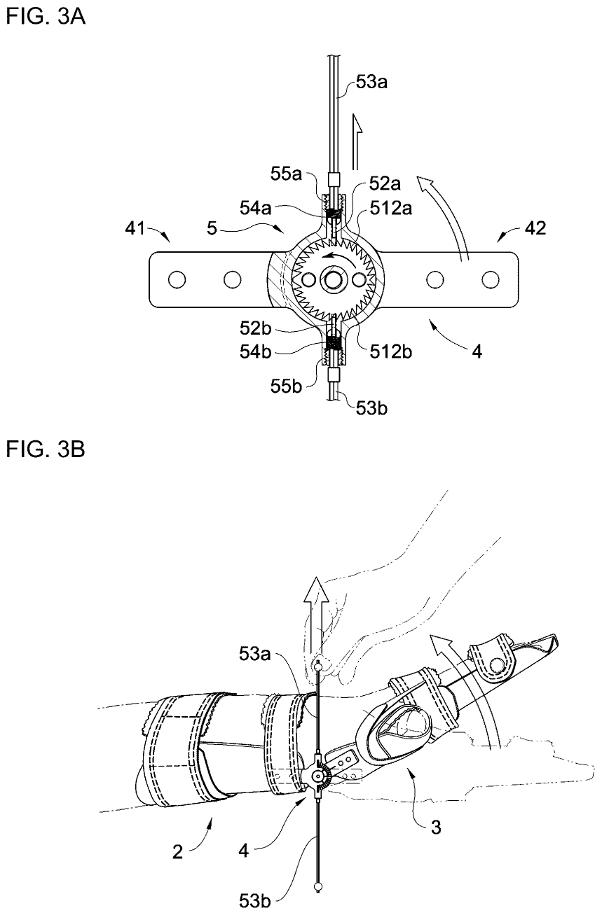

In a case in which the hand wearing portion 3 is rotated in the dorsiflexion direction from the above state, the first stopper 52a moves along the first slit 416a in the upward direction against the urging force of the first spring 54a by gripping and pulling the first wire 53a in the upward direction as shown in FIGS. 3A and 3B, and the engagement state between the first stopper 52a and the first ratchet teeth 512a is released. Note that, although the first spring 52a also moves in the upward direction along with the movement of the first stopper 54a, the first fastener 55a functions as a stopper, and therefore the first spring 54a is prevented from jumping out to the outside.

On the other hand, the engagement state between the second stopper 52b and the second ratchet teeth 512b is maintained, but the second ratchet teeth 512b have an inclination angle in the palmar flexion direction, and therefore, when the hand wearing portion 3 is turned in the dorsiflexion direction, the second stopper 52b slides on the front teeth of the second ratchet teeth 512b, so that the turning in the dorsiflexion direction is allowed. Furthermore, an operational feeling is obtained each time the second stopper 52b exceeds the apex of the second ratchet teeth 512b, and therefore the turning state can be sensorily grasped.

Then, when the first wire 53a gripped at a position at an arbitrary angle is released after the hand wearing portion 3 is turned in the dorsiflexion direction, the first stopper 52a is again engaged with the first ratchet teeth 512a by the urging force of the first spring 54a, and the hand wearing portion 3 is fixed in a state of being turned by a predetermined angle in the dorsiflexion direction.

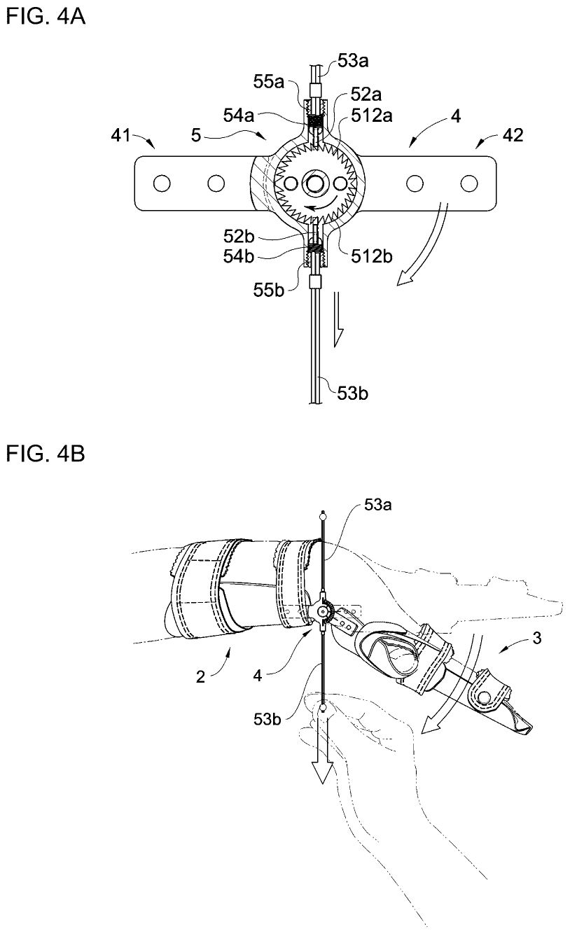

Next, when the hand wearing portion 3 is turned in the palmar flexion direction, the second stopper 52b moves in the downward direction along the second slit 416b against the urging force of the second spring 54b by gripping and pulling the second wire 53b toward the downward direction as shown in FIGS. 4A and 4B, and the engagement state between the second stopper 52b and the second ratchet teeth 512b is released. Note that, although the second spring 54b also moves in the downward direction along with the movement of the second stopper 52b, the second fastener 55b functions as a stopper, and therefore the second spring 54b is prevented from jumping out to the outside.

On the other hand, although the engagement state between the first stopper 52a and the first ratchet teeth 512a is maintained, the first ratchet teeth 512a have an inclination angle in the dorsiflexion direction, and therefore, when the hand wearing portion 3 is turned in the palmar flexion direction, the first stopper 52a slides on the front teeth of the first ratchet teeth 512a, and therefore the turning in the palmar flexion direction is allowed. Then, in the same way as the turning in the dorsiflexion direction, an operational feeling is obtained each time the first stopper 52a exceeds the apex of the first ratchet tooth 512a, and therefore the turning state can be sensorily grasped.

Subsequently, in the same way as the operation in the dorsiflexion direction when the second wire 53b gripped at a position at an arbitrary angle is released by turning the hand wearing portion 3 in the palmar flexion direction, the second stopper 52b is again engaged with the second ratchet teeth 512b by the urging force of the second spring 54b, and the hand wearing portion 3 is fixed in a state of being turned by a predetermined angle in the palmar flexion direction.

As described above, the state of engagement and disengagement between the first stopper 52a and the first ratchet tooth 512a and between the second stopper 52b and the second ratchet teeth 512b can be easily switched by operating the first wire 53a and the second wire 53b. Therefore, rehabilitation can be performed by arbitrarily setting the angle of the hand wearing portion 3 according to the recovery state of the affected limb on which the orthotic device 1 is worn.

Additionally, after the turning angle is set, the first stopper 52a and the second stopper 52b are again engaged with the first ratchet teeth 512a and the second ratchet teeth 512b, respectively, by releasing the first wire 53a and the second wire 53b, and the hand wearing portion 3 is fixed at a set arbitrary turning angle, and therefore, for example, even if an external force acts on the orthotic device 1, the turning angle does not unintentionally change, and therefore safety is also enhanced.

Although the orthotic device 1 according to the embodiment of the present invention has been described as above, a rotation restricting portion that restricts, for example, a turning range of the palmar flexion side and the dorsal flexion side of the hand wearing portion 3 may be provided. For example, by installing the rotation restricting portion at a predetermined position of the first connecting portion 41 and by allowing the second connecting portion 42 to come into contact with the rotation restricting portion when the second connecting portion 42 turns toward the palmar flexion side or the dorsal flexion side, further turning is restricted, and therefore safety in use can be further enhanced. The set position, the shape, and the like of the rotation restricting portion can be arbitrarily changed according to the turning range.

Additionally, although a specification has been described in which the first wire 53a is operated when the ratchet teeth are turned in the dorsiflexion direction, and the second wire 53b is operated when the ratchet teeth are turned in the palmar flexion direction in the above embodiment, the second wire 53b may be operated when the ratchet teeth are turned in the dorsiflexion direction, and the first wire 53a may be operated when the ratchet teeth are turned in the palmar flexion direction by assembling the ratchet gear 51 such that the ratchet teeth 512 are inclined in a direction opposite to that of the embodiment.

Furthermore, in the above embodiment, a case in which the joint portion 4 is applied to the orthotic device for assisting the movement of the wrist joint has been described, and yet the joint portion 4 can also be applied to, for example, an orthotic device for assisting the movement of the shoulder joint or the elbow joint. Furthermore, the present invention can be applied not only to an orthotic device for the upper limb but also to an orthotic device for the lower limb for assisting the movement of the ankle joint or the knee joint.

As described above, each of a joint for orthotic devices and an orthotic device to which the present invention is applied has a simple structure and is low in cost, and is practical for a patient.

### DESCRIPTION OF REFERENCE SIGNS

1 Orthotic device
2 Forearm wearing portion
21 Main body portion
22 First belt body
23 Second belt body
3 Hand wearing portion
31 Main body portion
32 Third belt body
33 Fourth belt body
34 Fifth belt body
4 Joint portion
41 First connecting portion
411 First base end portion
412 First head portion
413a,413b Through hole
414 Concave groove
415 Stopper holding portion
415a First stopper holding portion
415b Second stopper holding portion
416 Slit
416a First slit
416b Second slit
42 Second connecting portion
421 Second base end portion
422 Second head portion
423a,423b Through hole
424 Ridge portion
5 Shaft support portion
51 Ratchet gear
511 Main body portion
512 Ratchet teeth
512a First ratchet teeth
512b Second ratchet teeth
52 Stopper
52a First stopper
52b Second stopper
53 Wire
53a First wire
53b Second wire
54 Spring
54a First spring
54b Second spring
55 Fastener
55a First fastener
55b Second fastener

1. A joint for orthotic devices, the joint comprising:
a first connecting portion that includes a first base end portion and a first head portion, the first base end portion being connected to a first member;
a second connecting portion that includes a second base end portion and a second head portion, the second base end portion being connected to a second member; and
a shaft support portion that pivotally supports the first head portion of the first connecting portion and the second head portion of the second connecting portion with respect to each other so as to be rotatable in one rotation direction in which the second connecting portion moves upwardly from a reference state in which the first connecting portion and the second connecting portion become substantially parallel to a horizontal axis and in one other rotation direction in which the second connecting portion moves downwardly from the reference state, the shaft support portion including:
a ratchet gear including a plurality of ratchet teeth formed along an outer periphery of a substantially circular-shaped main body portion;
a first stopper that engages with the ratchet teeth and restricts rotation in the one rotation direction; and a second stopper that is located to face the first stopper and engages with the ratchet teeth and restricts rotation in the one other rotation direction;
in the reference state,
the ratchet teeth including:
a first ratchet tooth that is inclined in the one rotation direction in an upper half peripheral portion of the main body portion on a boundary of a diameter line substantially orthogonal to a rotation axis of the ratchet gear and that is engageable with the first stopper; and
a second ratchet tooth that is inclined in the other rotation direction in a lower half peripheral portion of the main body portion and that is engageable with the second stopper, and;
the first head portion or the second head portion including:
a first stopper holding portion in which a first spring that urges the first stopper in a direction of engaging with the first ratchet tooth and a first wire connected to the first stopper and extending upwardly from the first head portion or the second head portion in a direction opposite to an urging direction of the first spring are housed; and
a second stopper holding portion in which a second spring that urges the second stopper in a direction in which the second stopper engages with the second ratchet tooth and a second wire connected to the second stopper and extending downwardly from the first head portion or the second head portion in a direction opposite to an urging direction of the second spring are housed.

2. The joint for orthotic devices according to claim 1, wherein the first connecting portion includes a rotation restricting portion that restricts the one rotation direction of the second connecting portion and a rotation range in the one other rotation direction.

3. The joint for orthotic devices according to claim 1, wherein a concave portion in which the ratchet teeth are loosely fitted is formed in either one of the first head portion and the second head portion, and a protruding portion for fixing the ratchet teeth is provided on other one of the first head portion and the second head portion, and the first head portion and the second head portion are fitted together so as to form a same plane.

4. The joint for orthotic devices according to claim 1, wherein an angle formed between an imaginary straight line connecting an apex of the first ratchet tooth and a rotation axis of the ratchet gear and an inclined surface of a front tooth of the first ratchet tooth is approximately 40° to 45°, and
an angle formed between an imaginary straight line connecting an apex of the second ratchet tooth and a rotation axis of the ratchet gear and an inclined surface of a front tooth of the second ratchet tooth is approximately 40° to 45°.

5. An orthotic device comprising:
a forearm wearing portion that is worn on a forearm;
a hand wearing portion that is worn on a hand; and
a joint for orthotic devices that pivotally supports the hand wearing portion rotatably with respect to the forearm wearing portion;
the joint for orthotic devices including:
a first connecting portion including a first base end portion and a first head portion, the first base end portion being connected to a first forearm wearing portion;
a second connecting portion including a second base end portion and a second head portion, the second base end portion being connected to the hand wearing portion; and
a shaft support portion, the shaft support portion including:
a ratchet gear pivotally supporting the first head portion of the first connecting portion and the second head portion of the second connecting portion with respect to each other such that the second connecting portion is rotatable in a dorsiflexion direction from a reference state in which the first connecting portion and the second connecting portion are substantially parallel to a horizontal axis and such that the second connecting portion is rotatable in a palmar flexion direction from the reference state, the ratchet gear including a plurality of ratchet teeth formed along an outer periphery of a substantially circular-shaped main body;
a first stopper that engages with the ratchet teeth and that restricts rotation in the dorsiflexion direction; and
a second stopper that is located to face the first stopper and that engages with the ratchet teeth and that restricts rotation in the palmar flexion direction,
in the reference state,
the ratchet teeth including:
a first ratchet tooth that is inclined in a dorsiflexion direction in an upper half peripheral portion of the main body portion on a boundary of a diameter line substantially orthogonal to a rotation axis of the ratchet gear and that is engageable with the first stopper; and
a second ratchet tooth that is inclined in a palmar flexion direction in a lower half peripheral portion of the main body portion and that is engageable with the second stopper, and
the first head portion or the second head portion including:
a first stopper holding portion in which a first spring that urges the first stopper in a direction of engaging with the first ratchet tooth and a first wire connected to the first stopper and extending upwardly from the first head portion or the second head portion in a direction opposite to an urging direction of the first spring are housed; and
a second stopper holding portion in which a second spring that urges the second stopper in a direction of engaging with the second ratchet tooth and a second wire connected to the second stopper and extending downwardly from the first head portion or the second head portion in a direction opposite to an urging direction of the second spring are housed.
